(19) United States
(12) Patent Application Publication
(10) Pub. No.: US 2005/0220744 A1
(43) Pub. Date: Oct. 6, 2005

(54) Polysaccharide/functional compound complex

(75) Inventors: Motoaki Kamachi, Kanagawa (JP); Harumi Kamachi, Chiba (JP); Toyoji Kakuchi, Hokkaido (JP); Toshifumi Satoh, Hokkaido (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K.

(21) Appl. No.: 11/088,794

(22) Filed: Mar. 25, 2005

Related U.S. Application Data
(60) Provisional application No. 60/560,606, filed on Apr. 9, 2004.

(30) Foreign Application Priority Data
Mar. 31, 2004 (JP) 2004-105930

(51) Int. Cl. A61K 7/06

(52) U.S. Cl. 424/70.1

(57) ABSTRACT

The present invention provides a polysaccharide/functional compound complex comprising a functional compound and a multi-branched polysaccharide derivative, wherein the multi-branched polysaccharide derivative has a skeleton of a multi-branched polysaccharide composed of a saccharide as a structural unit, and at least one of the hydroxyl groups (OH) of the multi-branched polysaccharide is substituted by OR, wherein R represents a hydrogen atom, a hydrocarbon having 1 to 30 carbon atoms, or a hydrocarbon having 1 to 30 carbon atoms and hetero atom. The functional compound can be efficiently incorporated into polysaccharide derivative, and further by exposing the polysaccharide complex to an environment such as a solution, sustained release of the functional compound can be controlled. By utilizing this property, the complex can be efficiently used in external preparations for skin such as cosmetic products.

### CROSS-REFERENCE TO RELATED APPLICATIONS

This is an application filed pursuant to 35 U.S.C. Section 111(a) with claiming the benefit of U.S. provisional application Ser. No. 60/560,606 filed Apr. 9, 2004 under the provision of 35U.S.C. 111(b), pursuant to 35 U.S.C. Section 119(e) (1).

### TECHNICAL FIELD

The present invention relates to a novel polysaccharide/functional compound complex. More specifically, the present invention relates to a complex of a functional compound and a multi-branched polysaccharide derivative that controls the dissolution and elution of the functional compound, and by utilizing this property of controlling the dissolution and elution of the functional compound, the complex can be used, for example, in a cosmetic product as an external preparation for skin.

### BACKGROUND ART

It is a conventional technique to blend a combination of a saccharide and a functional compound into an external preparation for skin. For example, although β-lactose is often used in a cosmetic product, it has a low solubility at a low temperature, therefore in order to increase the solubility, it is necessary to process it into a fine powder or the like. A polysaccharide is often used in an external preparation for skin, and the typical example of a compound used in combination with a functional compound, other than polysaccharides, is cyclodextrin. In JP-A-2003-12439(“JP-A” means a publication of an unexamined Japanese patent application), an external preparation for skin in which cyclodextrin is used in order to prevent coloration of an ascorbic acid derivative and generation of an odor is disclosed. However, cyclodextrin imposes many restrictions on compounds to be included by cyclodextrin as follows: a compound that cyclodextrin can encapsulate has to be in a size that fits the inside diameter of cyclodextrin; and since the inside of cyclodextrin is a hydrophobic environment, a nonaqueous compound is generally selected. In addition, cyclodextrin having low solubility as compared with that of a water-soluble sugar chain, is not always easy to use.

In addition, other than a linear chain polysaccharide, a polysaccharide having a unique structure has been discovered recently, and it has been proposed that it be used in combination with a functional compound. For example, JP-A-2003-238447 discloses that glucan having a cyclic structure and a physiologically active component are mixed and used in an external preparation, and as the effect, the document only discloses that glucan mixed therein contributes to alleviating the strong stimulus of the physiologically active substance to the skin. However, since glucan having a cyclic structure is a naturally occurring substance, its molecular weight ranges widely, it is extremely difficult to control the structure and quality of products constant as compared with those of a synthetic compound.

On the other hand, as a synthetic polysaccharide, in JP-A-2003-252904 discloses a production method for obtaining an extremely unique multi-branched polysaccharide by ionic polymerization of an anhydrosaccharide as a monomer. Since it is a synthetic production method, conditions such as the molecular weight, branching degree, solubility or the like of the product can be regulated at a desired level. However, the blending of the product into an external preparation for skin and its efficacy are totally unknown.

### DISCLOSURE OF THE INVENTION

One of the objects of the present invention is to provide an effective complex of a polysaccharide and a functional compound. More specifically, the objects of the present invention are to provide a polysaccharide/functional compound complex which has a structure efficiently including a functional compound, and can release the functional compound at a desired rate by changing the external environment, and also to provide an external preparation for skin such as a cosmetic product which comprises the polysaccharide/functional compound complex.

The present inventors carried out intensive studies in order to solve the foregoing problems. As a result, they found out that variously changing the structure of a multi-branched polysaccharide derivative having a skeleton of a multi-branched polysaccharide composed of a saccharide as a structural unit enables formation of a structure which efficiently includes a functional compound, that by utilizing such a structure, a complex of a polysaccharide and a functional compound can be formed, that the functional compound is gradually released and separated from the polysaccharide/functional compound complex by exposing this polysaccharide/functional compound complex to an external environment which has been changed, and that as a result the feeling of use and effect of an external preparation for skin can be significantly improved by compounding this polysaccharide/functional compound complex in the product, and based on these findings, completed the present invention.

In other words, the present invention relates to the following polysaccharide/functional compound complex, external preparation for skin and cosmetic product.
1. A polysaccharide/functional compound complex comprising a functional compound and a multi-branched polysaccharide derivative, wherein the multi-branched polysaccharide derivative has a skeleton of a multi-branched polysaccharide composed of a saccharide as a structural unit, and at least one of the hydroxyl groups (OH) of the multi-branched polysaccharide is substituted by OR, wherein R represents a hydrogen atom, a hydrocarbon having 1 to 30 carbon atoms, or a hydrocarbon having 1 to 30 carbon atoms and hetero atom.
2. The polysaccharide/functional compound complex according to 1, wherein the multi-branched polysaccharide constituting the skeleton is a polymer composed of, as a monomer, an anhydrosaccharide and/or an anhydrosaccharide derivative in which at least one of the hydroxyl groups (OH) of the anhydrosaccharide has been substituted by OR, wherein R represents a hydrogen atom, a hydrocarbon having 1 to 30 carbon atoms, or a hydrocarbon having 1 to 30 carbon atoms and hetero atom.
3. The polysaccharide/functional compound complex according to 2, wherein the anhydrosaccharide or the anhydrosaccharide portion of the anhydrosaccharide derivative is at least one kind selected from the group consisting of 1,6-anhydrosaccharide, 1,4-anhydrosaccharide, 1,3-anhydrosaccharide, 1,2-anhydrosaccharide, and 5,6-anhydrosaccharide.
4. The polysaccharide/functional compound complex according to any one of 1 to 3, wherein the branching degree of the multi-branched polysaccharide is 0.05 to 1.00.
5. The polysaccharide/functional compound complex according to any one of 1 to 4, wherein the functional compound is a pigment compound.
6. The polysaccharide/functional compound complex according to any one of 1 to 4, wherein the functional compound is a compound having a physiological activity.
7. The polysaccharide/functional compound complex according to 6, wherein the compound having a physiological activity is at least one selected from the group consisting of raw material compounds for cosmetic products, raw material compounds for quasi-drug products and compounds for drug products.
8. The polysaccharide/functional compound complex according to 7, wherein the raw material compound for a cosmetic product is ascorbic acid, an ascorbate or an ascorbic acid derivative.
9. An external preparation for skin including 0.01 to 100% by mass of the polysaccharide/functional compound complex according to any one of 1 to 8.
10 A cosmetic product including 0.01 to 100% by mass of the polysaccharide/functional compound complex according to any one of 1 to 8.

Examples of the polysaccharide/functional compound complex of the present invention may include, for example, a complex formed with a multi-branched polysaccharide derivative and a functional compound by a physical action such as Van der Waals forces, a complex formed by a chemical action such as an ionic bond of the ions in a multi-branched polysaccharide derivative and a functional compound.

The multi-branched polysaccharide constituting the skeleton of the multi-branched polysaccharide derivative of the present invention is a compound composed of one kind or multiple kinds of saccharides as the structural components. The structure is a dendritic structure having many branching points wherein three or more sites binding with adjacent saccharide molecules exist in one saccharide molecule having multiple hydroxyl groups. Generally, a polysaccharide that exists in a variety of types as a natural substance is a linear polysaccharide or a branched polysaccharide having not so many branching points, and both types have its main chain. A multi-branched polysaccharide is a compound wherein its main chain cannot be clearly distinguished due to presence of many branching points.

Examples of the saccharide constituting the multi-branched polysaccharide may include, as a pentose, ribose and deoxyribose, and as a hexose, glucose, fructose and galactose.

As a production method of the multi-branched polysaccharide, a conventionally known method can be applied. As a production method by a chemical synthesis method, a production method of obtaining a multi-branched polysaccharide by polymerizing a derivative of a monosaccharide as a monomer can be exemplified. Examples thereof include, as disclosed in JP-A-2003-252904, a production method of obtaining a multi-branched polysaccharide by ionic polymerization using an anhydrosaccharide as a monomer.

In this case, examples of the anhydrosaccharide may include, for example, 1,6-anhydrosaccharide, 1,4-anhydrosaccharide, 1,3-anhydrosaccharide, 1,2-anhydrosaccharide, 5,6-anhydrosaccharide, etc., and a derivative thereof. Specific examples may include 1,6-anhydro-β-D-glucopyranose, 1,6-anhydro-β-D-mannopyranose, 1,6-anhydro-β-D-galactopyranose, 1,6-anhydro-β-D-allopyranose, 1,6-anhydro-β-D-altropyranose, 1,4-anhydro-β-D-ribopyranose, 1,4-anhydro-α-D-xylopyranose, 1,4-anhydro-α-L-arabinopyranose, 1,4-anhydro-α-D-lyxopyranose, 1,3-anhydro-α-D-glucopyranose, 1,3-anhydro-β-D-mannopyranose, 1,2-anhydro-α-D-glucopyranose, 1,2-anhydro-β-D-mannopyranose, 5,6-anhydro-α-D-glucopyranose, etc., and a derivative thereof.

In addition, as the ionic polymerization, cation polymerization and anion polymerization can be exemplified. Further, there is a production method of obtaining a multi-branched polysaccharide by using an animal, plant or microorganism, or by using a method of raising, cultivating or culturing it. In this case, a multi-branched polysaccharide with a desired branching degree can be directly used. However, as a method of producing a multi-branched polysaccharide whose branching degree has been increased, for example, by a production method as disclosed in JP-A-1996-41104, the branching degree can be increased so that a branched polysaccharide may become a multi-branched polysaccharide.

The multi-branched polysaccharide derivative of the present invention has a structure in which part and/or all of the hydroxyl groups in the multi-branched polysaccharide that constitutes the skeleton have been substituted by substituent(s). Examples include a compound in which at least one of the hydroxyl groups OH of the multi-branched polysaccharide has been substituted by OR wherein R represents a hydrogen atom, a hydrocarbon having 1 to 30 carbon atoms, or a hydrocarbon having 1 to 30 carbon atoms which has hetero atom.

Examples of the R may include, for example, a methyl group, ethyl group, propyl group, isopropyl group, butyl group, 1-methylpropyl group, 2-methylpropyl group, pentyl group, 1-methylbutyl group, 2-methylbutyl group, 3-methylbutyl group, 1-ethylpropyl group, hexyl group, 1-methylpentyl group, 2-methylpentyl group, 3-methylpentyl group, 4-methylpentyl group, 1-ethylbutyl group, 2-ethylbutyl group, heptyl group, 1-methylhexyl group, 2-methylhexyl group, 3-methylhexyl group, 4-methylhexyl group, 5-methylhexyl group, 1-ethylpentyl group, 2-ethylpentyl group, 3-ethylpentyl group, octyl group, 1-methylheptyl group, 2-methylheptyl group, 3-methylheptyl group, 4-methylheptyl group, 5-methylheptyl group, 6-methylheptyl group, 1-ethylhexyl group, 2-ethylhexyl group, 3-ethylhexyl group, 4-ethylhexyl group, 1-propylpentyl group, 2-propylpentyl group, nonyl group, decyl group, undecyl group, 10-undecenyl group, dodecyl group, tridecyl group, tetradecyl group, pentadecyl group, hexadecyl group, 9-hexadecenyl group, heptadecyl group, octadecyl group, isostearyl group, cis-9-octadecenyl group, 11-octadecenyl group, cis-cis-9,12-octadecadienyl group, 9,12,15-octadecatrienyl group, 6,9,12-octadecatrienyl group, 9,11,13-octadecatrienyl group, nonadecyl group, 2,6,10,14-tetramethyl pentadecyl group, icosanyl group, 8,11-icosadienyl group, 5,8,11-icosatrienyl group, 5,8,11,14-icosatetraenyl group, 3,7,11,15-tetramethylhexadecyl group, heneicosanyl group, docosanyl group, acetyl group, propionyl group, isopropionyl group, butyryl group, 1-methylpropionyl group, 2-methylpropionyl group, valeryl group, 1-methylbutyryl group, 2-methylbutyryl group, 3-methylbutyryl group, 1-ethylpropionyl group, hexanoyl group, 2-methylpentanoyl group, 3-methylpentanoyl group, 4-methylpentanoyl group, 2-ethylbutanoyl group, heptanoyl group, 2-methylhexanoyl group, 3-methylhexanoyl group, 4-methylhexanoyl group, 2-ethylpentanoyl group, 3-ethylpentanoyl group, octanoyl group, 2-methylheptanoyl group, 3-methylheptanoyl group, 4-methylheptanoyl group, 5-methylheptanoyl group, 6-methylheptanoyl group, 2-ethylhexanoyl group, 3-ethylhexanoyl group, 4-ethylhexanoyl group, 2-propylpentanoyl group, nonanoyl group, decanoyl group, undecanoyl group, 10-undecenoyl group, dodecanoyl group, tridecanoyl group, tetradecanoyl group, pentadecanoyl group, hexadecanoyl group, 9-hexadecenoyl group, heptadecanoyl group, octadecanoyl group, isostearyl group, cis-9-octadecenoyl group, 11-octadecenoyl group, cis,cis-9,12-octadecadienoyl group, 9,12,15-octadecatrienoyl group, 6,9,12-octadecatrienoyl group, 9,11,13-octadecatrienoyl group, nonadecanoyl group, 2,6,10,14-tetramethylpentadecanoyl group, icosanoyl group, 8,11-icosadienoyl group, 5,8,11-icosatrienoyl group, 5,8,11,14-icosatetraenoyl group, 3,7,11,15-tetramethylhexadecanoyl group, heneicosanoyl group, docosanoyl group, phosphatidyl group and the like. Further, a compound in which R has been bound with an isocyanate group and the like are also included.

As the synthetic method of the multi-branched polysaccharide derivative of the present invention, for example, a method of synthesizing a multi-branched polysaccharide derivative by modifying a hydroxyl group of a saccharide with a desired functional group after obtaining a multi-branched polysaccharide for constituting the skeleton, or a method of synthesizing a multi-branched polysaccharide derivative by polymerization after modifying a hydroxyl group of an anhydrosaccharide with a desired functional group to serve as a monomer of a multi-branched polysaccharide may be employed.

With regard to the modification of the hydroxyl group, for example, a method by a common esterification reaction using a carboxylic acid derivative can be employed. Specific examples include a method of performing reaction by using an acid chloride or a mixed acids anhydride prepared from corresponding carboxylic acid(s), a method of performing reaction by using carbodiimide, a method of using an acid anhydride and the like. Further, in the case of a compound having an isocyanate group, a modification method of forming an urethane bond by heating or by using a catalyst such as a tin-based catalyst or an amine-based catalyst, and the like can be employed.

The branching degree of the multi-branched polysaccharide constituting the skeleton of the multi-branched polysaccharide derivative of the present invention is 0.05 to 1.00, preferably 0.2 to 1.0, more preferably 0.4 to 1.0. The multi-branching degree in this case is generally calculated with Frechet formula as shown below.
Branching Degree=(number of dendritic units+number of polymer ends)/(number of dendritic units+number of polymer ends+number of linear chain units)

The functional compound that can be used in the present invention is not particularly limited as long as it has a functionality, however, examples include a pigment compound and a compound having a physiological activity.

Examples of the pigment compound include, for example, rose bengal, thymol blue, methyl orange, alizarin yellow, nile red, rhodamine, merocyanine, congo red, lycopene, beta-carotene, canthaxanthin, asthaxanthin, gardenia pigment, safflower pigment, turmeric pigment, monascus pigment, paprika pigment, beet red, cochineal, shikonin, anthocyanin, carminic acid, laccaic acid, carsamine, brazilin, crocin, Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5,. Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 205, Orange No. 207, nitro pigment, azo pigment, nitroso pigment, triphenylmethane pigment, xanthene pigment, quinoline pigment, anthraquinone pigment, indigo pigment, pyrene dye, phthalocyanine pigment, sodium copper chlorophyllin, riboflavin butyric acid ester, flavonoid, quinone, porphyrin, water-soluble annatto, squid ink powder, caramel, guaiazulene and the like.

Examples of the compound having physiological activity include compounds serving as raw material for cosmetics, compounds serving as rawmaterial for quasi-drug products and compounds for drug products .

Further, examples of compounds serving as raw material for cosmetics and compounds serving as raw material for quasi-drug products include compounds described in, for example, Keshouhin genryou kizyun (Standards of raw materials of cosmetics), second edition, notes, edited by Society of Japanese Pharmacopoeia, 1984 (YAKUJI NIPPO LIMITED.), Keshouhin genryou kizyun-gai seibun kikaku (Standards of raw materials of cosmetics, nonstandard ingredients), under the editorship of Pharmaceutical Affairs Bureau Evaluation and Registration Division, 1993 (YAKUJI NIPPO LIMITED.), Keshouhin genryou kizyun-gai seibun kikaku tsuiho (Standards of raw materials of cosmetics, nonstandard ingredient Supplement), under the editorship of Pharmaceutical Affairs Bureau Evaluation and Registration Division, 1993 (YAKUJI NIPPO LIMITED.), Keshouhin syubetsu kyoka kizyun (Standards of cosmetic classification permission), under the editorship of Pharmaceutical Affairs Bureau Evaluation and Registration Division, 1993 (YAKUJI NIPPO LIMITED.), Keshouhin syubetsu haigou seibun kikaku (Standards of cosmetic classification ingredients), under the editorship of Pharmaceutical Affairs Bureau Evaluation and Registration Division, 1997 (YAKUJI NIPPO LIMITED.), Keshouhin genryou jiten (Dictionary of raw materials of cosmetics), 1991 (Nikko Chemicals Co., Ltd.), Atarashii Keshouhinkinou Sozai 300 (New 300 raw materials having cosmetic functions), 2002 (CMC Publishing Co., Ltd) and the like.

Examples of the method of blending the polysaccharide/functional compound complex to be used in the present invention into an external preparation for skin include a method of producing an external preparation for skin by mixing as a solid, powder or semisolid, a method of producing an external preparation for skin by mixing as an aqueous solution, a method of producing an external preparation for skin by mixing as a solution of an alcohol or an appropriate solvent, and a method of producing an external preparation for skin by mixing or adding by other known methods.

The polysaccharide/functional compound complex to be used in the present invention may be isolated as, for example, a form of powder and blended in an external preparation for skin and also, a solution obtained in the process of the production may be directly blended in an external preparation for skin.

The polysaccharide/functional compound complex to be used in the present invention, as long as the blending amount does not hinder formation of the external preparation for skin, can be blended in an amount of 0.01% by mass to 100% by mass, preferably from 0.1% by mass to 80% by mass, more preferably at 1% by mass to 50% by mass. If the blending amount is less than 0.01% by mass based on the external preparation for skin, adhesiveness and affinity to the skin are not sufficient, and a feeling such as a moisturizing feeling to the skin cannot be obtained sufficiently in some cases. It can be blended within such a range that essential functions of the external preparation for skin may not be impaired.

The term “external preparation for skin” used in the present invention includes cosmetics, detergents, bath agents, soaps and the like which are used in direct contact with the skin.

The external preparation for skin of the present invention may be any type as long as it is used in contact with the skin. Examples of the external preparation for skin of the present invention include in a wide sense, for example, skin milk, skin cream, foundation cream, massage cream, cleansing cream, shaving cream, cleansing foam, skin lotion, lotion, facial mask, lip rouge, rouge, eye shadow, manicure, soap, body shampoo, hand soap, shampoo, hair conditioner, hair tonic, hair treatment, hair cream, hair spray, hair growth tonic, baldness remedy, hair dye, shmaltz, hair remover, anti-dandruff lotion, toothpaste, artificial teeth adhesive, gargle, permanent wave agent, curling agent, styling agent, ointment, cataplasm, tape, bath agent, adiaphoretic, sun protectant and the like.

In particular, a preferred use of the external preparation for skin of the present invention is a cosmetic product. In addition, the user may be anyone regardless of sex or age. Further, products to be used in contact with the skin of animals other than human are also included.

Examples of the form of the external preparation for skin of the present invention include many forms such as powder, granule, tablet, gel and foam, as well as solid, liquid, semisolid and gas.

In the external preparation for skin of the present invention, as a component other than the polysaccharide/functional compound complex, components generally used in an external preparation for skin can be blended within the range that does not impair the effect of this invention, as needed. Examples of components include compounds blendable in cosmetic products, compounds serving as raw materials for quasi-drug products, drug products and medicinal additives.

Specific examples of such an ingredient include hydrocarbons such as ozokerite, α-olefin oligomer, light isoparaffin, light liquid isoparaffin, squalene, squalane, synthetic squalane, phytosqualane, ceresin, paraffin, polyethylene powder, polybutene, microcrystalline wax, liquid isoparaffin, liquid paraffin, mineral oil and vaseline;

natural waxes such as jojoba oil, carnauba wax, candelilla wax, rice bran wax, shellac, lanolin, mink sebaceous wax, spermaceti wax, sugarcane wax, sperm whale oil, beeswax and montan wax, natural fats and fatty oils such as avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cotton seed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia jeponica oil, Camellia kissi oil, cacao butter, Japan wax, beef bone tallow, nest's-foot oil, swine tallow, equine tallow, ovine tallow, shea butter, macadamia nut oil and meadowfoam seed oil;

fatty acids such as lauric acid, myristic acid, palmitic acid, stearic acid, behenic acid, oleic acid, linoleic acid, linolenic acid, γ-linolenic acid, isostearic acid, 12-hydroxystearic acid, undecylenic acid and coconut oil fatty acid;

higher alcohols such as isostearyl alcohol, octyl dodecanol, hexyl decanol, cholesterol, phytosterol, lauryl alcohol, myristyl alcohol, cetyl alcohol, stearyl alcohol, oleyl alcohol, behenyl alcohol and cetostearyl alcohol;

alkylglyceryl ethers such as batyl alcohol, chimyl alcohol, serachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, propylene glycol dicaprate, propylene glycoldioleate, neopentyl glycol dicaprate, neopentyl glycol dioctanoate, glyceryl tricaprylate, glyceryl tri 2-ethyl hexanoate, glyceryl tricaprylate/tricaprate, glyceryl tricaprylate/tricaprate/tristearate, glyceryl triundecylate, glyceryl triisopalmitate, glyceryl triisostearate, trimethylolpropane tri 2-ethylhexanoate, trimethylolpropane triisostearate, pentaerythrityl tetra 2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di 2-ethylhexyl succinate, diisopropyl adipate, diisobutyl adipate, dioctyl adipate, diheptylundecyl adipate, sebacate diethyl, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methyl hydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane,
dimethylsiloxane-methyl(polyoxyethylene)siloxane-methyl(polyoxypropylene)siloxane copolymer,
dimethylsiloxane-methyl(polyoxyethylene)siloxane copolymer,
dimethylsiloxane-methyl(polyoxypropylene)siloxane copolymer,
dimethylsiloxane-methylcetyl oxysiloxane copolymer,
dimethylsiloxane-methyl stearoxysiloxane copolymer, polyether modified silicone, alcohol modified silicone, alkyl modified silicone and amino modified silicone;

polyhydric alcohols such as ethylene glycol, diethylene glycol, triethylene glycol, polyethylene glycol, propylene glycol, dipropylene glycol, polypropylene glycol, pentanediol, glycerin, diglycerin, polyglycerin, isoprene glycol, 1,3-butylene glycol, 3-methyl-1,3-butanediol, 1,3-butanediol, 1,2-pentanediol and 1,2-hexanediol;

saccharides such as mannitol, sorbitol, xylitol, maltitol, erythritol, pentaerythritol, glucose, sucrose, fructose, lactose, maltose, xylose and trehalose;

polymers such as sodium alginate, carrageen, agar, furcellaran, guar gum and carrageenan quince seed, Amorphophalus konjak (arum root) mannan, tamarind gum, tara gum, dextrin, starch, locust bean gum, gum arabic, gumgatti, karaya gum, gum tragacanth, arabinogalactan, pectin, quince, chitosan, starch, curdlan, xanthan gum, gellan gum, cyclodextrin, dextran, pullulan, microcrystalline cellulose, methyl cellulose, ethyl cellulose, hydroxyethyl cellulose, hydroxypropyl cellulose, hydroxypropylmethyl cellulose, carboxymethyl cellulose, carboxy starch, cationized cellulose, starch phosphate ester, cationized guar gum, carboxymethyl-hydroxypropylated guar gum, hydroxypropylated guar gum, albumin, casein, gelatin, sodium polyacrylate, polyacrylic amide, carboxyvinyl polymer, polyethylene imine, highly polymerized polyethylene glycol, polyvinyl alcohol, polyvinyl pyrrolidone, polyvinyl ether, polyacryl amide, acrylic acid copolymer, methacrylic acid copolymer, maleic acid copolymer, vinylpyridine copolymer, ethylene/acrylic acid copolymer, vinyl pyrrolidone based polymer, vinyl alcohol/vinyl pyrrolidone copolymer, nitrogen-substituted acrylamide based polymer, amino modified silicone, cationized polymer, dimethylacryl ammonium based polymer, acrylic acid based anion polymer, methacrylic acid based anion polymer, modified silicone, acrylate/methacrylate alkyl (C 10 to 30) copolymer and polyoxyethylene/polyoxypropylene copolymer;

alcohols such as ethanol, isopropyl alcohol, 1-butanol, 2-butanol and benzyl alcohol;

anionic surfactants such as coconut oil fatty acid potassium, coconut oil fatty acid sodium, coconut oil fatty acid triethanolamine, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassiumoleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calciummyristate, magnesiummyristate, aluminumdimyristate, aluminum isostearate, polyoxyethylene lauryl ether acetate, sodium polyoxyethylene lauryl ether acetate, polyoxyethylene tridecyl ether acetate, sodiumpolyoxyethylene tridecyletheracetate, sodiumstearoyl lactate, sodium isostearoyl lactate, sodium lauroyl sarcosine, coconut oil fatty acid sarcosine, sodium coconut oil fatty acid sarcosine, coconut oil fatty acid sarcosine triethanolamine, lauroyl sarcosine, potassium lauroyl sarcosine, lauroyl sarcosine triethanolamine, oleoyl sarcosine, sodium myristoyl sarcosine, sodium stearoyl glutamate, coconut oil fatty acid acyl glutamic acid, potassium coconut oil fatty acid acyl glutamate, sodium coconut oil fatty acid acyl glutamate, coconut oil fatty acid acyl glutamate triethanolamine, lauroylacyl glutamic acid, potassium lauroylacyl glutamate, sodium lauroylacyl glutamate, lauroylacyl glutamate triethanolamine, myristoylacyl glutamic acid, potassium myristoylacyl glutamate, sodium myristoylacyl glutamate, stearoylacyl glutamic acid, potassium stearoylacyl glutamate, disodium stearoylacyl glutamate, sodium hydrogenated beef tallow fatty acid acyl glutamate, sodium coconut oil fatty acid/hydrogenated beef tallow fatty acid acyl glutamate, sodium coconut oil fatty acid methylalanine, lauroyl methylalanine, sodium lauroyl methylalanine, lauroyl methylalanine triethanolamine, sodium myristoyl methylalanine, sodium lauroyl methyltaurine, potassium coconut oil fatty acid methyltaurine, sodium coconut oil fatty acid methyltaurine, magnesium coconut oil fatty acid methyltaurine, sodium myristoyl methyltaurine, sodium palmitoyl methyltaurine, sodium stearoyl methyltaurine, sodium oleoyl methyltaurine, sodium alkane sulfonate, sodium tetradecenesulfonate, sodium sulfosuccinatedioctyl, disodium lauryl sulfosuccinate, sodium coconut oil fatty acid ethyl ester sulfonate, sodiumlauryl sulfate, triethanolamine lauryl sulfate, sodiumcetyl sulfate, triethanolamine alkyl (11,13,15) sulfate, sodium alkyl (12,13) sulfate, triethanolamine alkyl (12,13) sulfate, alkyl (12,14,16) ammonium sulfate, diethanolamine alkyl (12 to 13) sulfate, triethanolamine alkyl (12 to 14) sulfate, triethanolamine alkyl (12 to 15) sulfate, magnesium coconut oil alkyl sulfate/triethanolamine, lauryl ammonium sulfate, potassium lauryl sulfate, magnesium lauryl sulfate, monoethanolamine lauryl sulfate, diethanolamine lauryl sulfate, sodium myristyl sulfate, sodium stearylsulfate, sodium oleyl sulfate, triethanolamine oleyl sulfate, sodiumpolyoxyethylene lauryl ether sulfate, triethanolamine polyoxyethylene lauryl ether sulfate, sodium polyoxyethylene (1) alkyl (11,13,15) ether sulfate, triethanolamine polyoxyethylene (1) alkyl (11,13,15) ether sulfate, sodium polyoxyethylene (3) alkyl (11 to 15) ether sulfate, sodium polyoxyethylene (2) alkyl (12,13) ethersulfate, sodiumpolyoxyethylene (3) alkyl (12 to 14) ether sulfate, sodium polyoxyethylene (3) alkyl (12to 15) ethersulfate, sodiumpolyoxyethylene (2) lauryl ether sulfate, sodium polyoxyethylene (3) myristyl ether sulfate, sodium higher fatty acid alkanol amide sulfate ester, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphate, sodium polyoxyethylene alkylphenyl ether phosphate, triethanolamine polyoxyethylene alkylphenyl ether phosphate, polyoxyethylene octyl ether phosphate, polyoxyethylene (10) alkyl (12,13) ether phosphate, polyoxyethylene alkyl (12 to 15) ether phosphate, polyoxyethylene alkyl (12 to 16) ether phosphate, triethanolamine polyoxyethylene lauryl ether phosphate and diethanolamine polyoxyethylene oleyl ether phosphate;

cationic surfactants such as dioctylamine, dimethylstearylamine, trilaurylamine, diethylaminoethylamide stearate, lauryl trimethylammonium chloride, cetyl trimethylammonium chloride, cetyl trimethylammoniumbromide, cetyl trimethylammonium saccharin, stearyl trimethylammoniumchloride, alkyl (20to 22) trimethylammoniumchloride, lauryl trimethylammonium bromide, alkyl (16,18) trimethylammonium chloride, stearyl trimethylammonium bromide, stearyl trimethylammonium saccharin, alkyl (28) trimethylammonium chloride, di(polyoxyethylene) oleyl methylammonium (2EO) chloride, dipolyoxyethylene stearyl methylammonium chloride, polyoxyethylene (1) polyoxypropylene (25) diethylmethylammonium chloride, tri(polyoxyethylene) stearyl ammonium (5EO) chloride, distearyl dimethylammoniumchloride, dialkyl (12 to 15) dimethylammoniumchloride, dialkyl (12 to 18) dimethylammonium chloride, dialkyl (14 to 18) dimethylammonium chloride, dicocoyl dimethylammonium chloride, dicetyl dimethylammonium chloride, isostearyllauryl dimethylammonium chloride, benzalkonium chloride, myristyl dimethylbenzyl ammonium chloride, lauryl dimethyl(ethylbenzyl) ammonium chloride, stearyl dimethylbenzyl ammonium chloride, lauryl pyridinium chloride, cetyl pyridinium chloride, lauroyl cholamino formylmethyl pyridinium chloride, stearoyl cholamino formylmethyl pyridinium chloride, alkyl isoquinolinium bromide, methyl benzethonium chloride and benzethonium chloride;

ampholytic surfactants such as 2-alkyl-N-carboxymethyl-N-hydroxyethyl imidazolium betaine, alkyldiamino ethyl glycine hydrochloride, sodium lauryldiamino ethyl glycine, sodium undecyl hydroxyethyl imidazolium betaine, undecyl-N-carboxymethyl imidazolium betaine, disodium coconut oil fatty acid acyl-N-carboxyethyl-N-hydroxyethyl ethylenediamine, disodium coconut oil fatty acid acyl-N-carboxyethoxyethyl-N-carboxyethyl ethylenediamine, disodium coconut oil fatty acid acyl-N-carboxymethoxyethyl-N-carboxymethyl ethylenediamine, sodium laurylamino propionate, sodium laurylamino dipropionate, triethanolamine laurylamino propionate, sodium palm oil fatty acid acyl-N-carboxyethyl-N-hydroxyethyl ethylenediamine, betaine lauryldimethylamino acetate, betaine coconut oil alkyldimethylamino acetate, betaine stearyl dimethylamino acetate, sodium stearyldimethyl betaine, coconut oil fatty acid amidopropyl betaine, palm oil fatty acid amidopropyl betaine, amidopropyl acetate betaine laurate, amidopropyl betaine ricinoleate, stearyl dihydroxyethyl betaine and lauryl hydroxysulfobetaine;

nonionic surfactants such as polyoxyethylene (10) alkyl (12,13) ether, polyoxyethylene lauryl ether, polyoxyethylene cetyl ether, polyoxyethylene stearyl ether, polyoxyethylene oleyl ether, polyoxyethylene (3,7,12) alkyl (12 to 14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2,10,20) isostearyl ether, polyoxyethylene oleylcetyl ether, polyoxyethylene (20) arachyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1,2,4,8) cetyl ether, polyoxyethylene (5) polyoxypropylene (1,2,4,8) cetyl ether, polyoxyethylene (10) polyoxypropylene (1,2,4,8) cetyl ether, polyoxyethylene (20) polyoxypropylene (1,2,4,8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifying ethylene glycol monostearate, diethylene glycol laurate, polyethylene glycol myristate, polyethylene glycol palmitate, diethylene glycol stearate, self-emulsifying polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, polyoxyethylene (20) coconut oil fatty acid sorbitan, polyoxyethylene (10 to 80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, lipophilic glycerin monostearate, lipophilic glycerin monooleate, self-emulsifying glycerin monostearate, coconut oil fatty acid glyceryl, glycerin laurate, glyceryl myristate, glyceryl isostearate, glyceryl ricinoleate, glyceryl monohydroxystearate, glycerin oleate, glyceryl linoleate, glyceryl erucate, glyceryl behenate, wheat germ oil fatty acid glyceride, safflower oil fatty acid glyceryl, hydrogenated soybean fatty acid glyceryl, saturated fatty acid glyceride, cotton seed oil fatty acid glyceryl, monomyristate glyceryl monoisostearate, mono tallowate glyceride, monolanolin fatty acid glyceryl, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, coconut oil fatty acid sorbitan, sorbitan isostearate, sorbitan sesquiisostearate, sorbitan distearate, diglyceryl isopalmitate, poly (4 to 10) glycerylmonolaurate, poly(10)glyceryl monomyristate, poly(2 to 10)glyceryl monostearate, poly(2 to 10) glyceryl monoisostearate, poly(2to 10) glyceryl monooleate, diglyceryl sesquioleate, poly(2 to 10)glyceryl diisostearate, poly(6 to 10)glyceryl distearate, diglyceryl triisostearate, poly(10)glyceryl tristearate, poly(10)glyceryl trioleate, poly (2) glyceryl tetraisostearate, decaglyceryl pentastearate, poly (6 to 10)glyceryl pentaoleate, poly(10)glyceryl heptastearate, decaglyceryl decastearate, poly (10) glyceryl decaoleate, concentrated poly(6)glyceryl ricinoleate, sucrose fatty acid ester, coconut oil fatty acid sucrose ester, alkyl glucoside, coconut oil alkyl dimethylamine oxide, lauryl dimethylamine oxide, dihydroxyethyl lauryl dimethylamine oxide, stearyl dimethylamine oxide, oleyl dimethylamine oxide and polyoxyethylene coconut oil alkyl dimethylamine oxide;

natural surfactants such as saponin, lecithin, soybean phospholipid, hydrogenated soybean phospholipid, soybean lysophospholipid, hydrogenated soybean lysophospholipid, yolk lecithin, hydrogenated yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosyl erythritol lipid;

ultraviolet ray absorbers such as: para-aminobenzoic acid derivatives such as para-aminobenzoic acid, ethyl para-aminobenzoate, glyceryl para-aminobenzoate, amyl para-dimethyl aminobenzoate and 2-ethylhexyl para-dimethyl aminobenzoate; cinnamic acid derivatives such as benzyl cinnamate, mono-2-ethyl hexanoate glyceryl dipara-methoxycinnamate, methyl 2,4-diisopropyl cinnamate, ethyl 2,4-diisopropyl cinnamate, potassium para-methoxycinnamate, sodium para-methoxycinnamate, isopropylpara-methoxycinnamate, 2-ethylhexyl para-methoxycinnamate, 2-ethoxyethyl para-methoxycinnamate and ethyl para-ethoxycinnamate; urocanic acid derivatives such as urocanic acid and ethyl urocanate; benzophenone derivatives such as 2,4-dihydroxybenzophenone, 2,2′,4,4′-tetrahydroxybenzophenone, sodium 2-hydroxy-4-methoxy-5-sulfobenzophenone, 2-hydroxy-4-methoxybenzophenone-5-sulfonate, 2-hydroxy-4-methoxybenzophenone, 2,2′-dihydroxy-4,4′-dimethoxybenzophenone and sodium 2,2′-dihydroxy-4,4′-dimethoxy-5-sulfobenzophenone; salicylic acid derivatives such as ethylene glycol salicylate, salicylate-2-ethylhexyl, phenyl salicylate, benzyl salicylate, p-tert-butylphenyl salicylate, homomenthyl salicylate and salicylate-3,3,5-trimethylcyclohexyl; 2-(2′-hydroxy-5′-methoxyphenyl)benzotriazole and 4-tert-butyl-4′-methoxybenzoyl methane;

powders and color materials such as: kaolin, silicic anhydride, magnesium aluminum silicate, sericite, talc, boron nitride, mica, montmorillonite, hemp cellulose powder, wheat starch, silk powder, maize starch; natural dyes such as nitro dyes, azo dyes, nitroso dyes, triphenylmethane dyes, xanthene dyes, quinoline dyes, anthraquinone dyes, indigo dyes, pyrene dyes, phthalocyanine dyes, flavonoid, quinone, porphyrin, water soluble annatto, sepia powder, caramel, guaiazulene, gardenia blue, gardenia yellow, cochineal, shikonin, sodium copper chlorophyllin, paprika dye, safflower red, safflower yellow, laccaic acid and riboflavin butyrate ester; carbon black, yellow iron oxide, black iron oxide, red iron oxide, iron blue, ultramarine blue, zinc oxide, chromiumoxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment.

plant extracts such as Angelica keiskei extract., Uncaria gambir extract, avocado extract, sweet hydrangea leaf extract, Gynostemma pentaphyllum makino extract, Althaea officinalis extract, Arnica montana extract, oil soluble Arnica montana extract, almond extract, aloe extract, Japanese styrax benzoin extract, Ginkgo biloba extract, Stinging nettle extract, Orris rhizome root extract, fennel extract, turmeric extract, dog rose fruit extract, Echinacea leaf extract, Scutellaria root extract, Phellodendron bark extract, Japanese captis extract, barley extract, okura extract, Hypericum perforatum extract, oil soluble Hypericum perforatum extract, Lamium album extract, oil soluble Lamium album extract, Ononis spinosa root extract, Nasturtium officinale extract, orange extract, orange flower water, seaweed extract, persimmon tannin, pueraria root extract, Japanese valerian extract, cattail extract, Chamomile (matricaria) extract, oil soluble Chamomile (matricaria) extract, Chamomile (matricaria) distillate, Avena sativa (oat) kernel extract, carrot extract, oil soluble carrot extract, carrot oil, Artemicia capillaris extract, Glycyrrhiza glabra (licorice) extract, powdered Glycyrrhiza glabra (licorice) extract, Glycyrrhiza glabra (licorice) extract flavonoid, cantharides tincture, raspberry extract, kiwi extract, cinchona extract, cucumber extract, apricot kernel extract, quince seed extract, gardenia florida extract, Sasa albomarginata extract, Sophora root extract, walnut shell extract, Citrus paradisi (grapefruit) extract, Clematis vitalba leaf extract, black sugar extract, chlorella extract, mulberrybark extract, Cinnamon bark extract, Gentian extract, Geranium herb extract, black tea extract, Nuphar extract, burdock root extract, oil soluble burdock root extract, wheat germ extract, hydrolyzed wheat powder, rice bran extract, fermented rice bran extract, Symphytum officinale (comfrey) extract, Asiasarum root extract, Crocus sativus (saffron) extract, Saponaria officinalis extract, oil soluble salvia extract, Crataegus cuneata fruit extract, Zanthoxylum fruit extract, Lentinus edodes extract, powdered Lentinus edodes extract, Rehmannia root extract, Lithospermum root extract, oil soluble Lithospermum root extract, Perilla herb extract, linden extract, oil soluble Tilia europaea extract, Filipendula extract, Peony root extract, Coix lacryma- jobi extract, ginger extract, oil soluble ginger extract, ginger tincture, Acorus calamus root extract, Betula pendula (birch) extract, oil soluble Betula alba (birch) extract, Betula pendula (birch) sap, Lonicera japonica extract, Equisetum arvense extract, oil soluble Equisetum arvense extract, scordinin, stevia extract, ivy extract, Crataegus oxyacantaha (whitethorn) extract, sambucus extract, Juniperus communis extract, Achillea milefolium extract, oil soluble Achillea milefolium extract, Menthapiperita (peppermint) extract, Salvia officinalis (sage) extract, oil soluble Salvia officinalis (sage) extract, Salvia officinalis (sage) water, Malva Sylvestris (mallow) extract, Apium graveolens (celery) extract, Cnidium officinale extract, Cnidium officinale water, Swertia herb extract, Glycine max (soybean) extract, Jujube extract, thyme extract, green tea extract, tea leaf dry distilled solution, tea seed extract, clove extract, Citrus unshiu peel extract, Camellia japonica extract, Centella asiatica extract, oil soluble walnut extract, duku extract, Terminalia sericea extract, Capsicum tincture, Japanese angelica root extract, oil soluble Japanese angelica root extract, Japanese angelica root water, Calendula officinalis flower extract, oil soluble Calendula officinalis flower extract, soy milk powder, peach seed extract, Bitter orange peel extract, Houttuynia cordata extract, Solanum lycopersicum (tomato) extract, Potentilla tormentilla Schrk (Rosaceae) extract, fermented soybeans extract, Ginseng extract, oil soluble Ginseng extract, Allium sativum (garlic) extract, wild rose extract, oil soluble wild rose extract, malt extract, malt root extract, Ophiopogon tuber extract, parsley extract, rye leaf juice concentrate, peppermint distillate, witch hazel distillate, witch hazel extract, rose extract, parietaria extract, Isodonis japonicus extract, Eriobotrya japonica leaf extract, oil soluble Eriobotrya japonica leaf extract, coltsfoot extract, hoelen extract, Ruscus aculeatus root extract, powdered Ruscus aculeatus root extract, grape extract, grape leaf extract, grape water, Hayflower extract, Luffa cylindrica fruit extract, Luffa cylindrica fruitwater, Carthamustinctorius (safflower) extract, oil soluble Tilia platyphyllos extract, linden distillate, Paeonia suffruticosa (peony) extract, Humulus lupulus (hops) extract, oil soluble Humulus lupulus (hops) extract, pine extract, Silybum marianum (milk thistle) extract, Aesculus hippocastanum (horse chestnut) extract, oil soluble Aesculus hippocastanum (horse chestnut) extract, Sapindus mukurossi extract, Melissa officinalis (balm mint) extract, Melilotus officinalis (melilot) extract, Prunus persica (peach) leaf extract, oil soluble Prunus persica (peach)leaf extract, bean sprouts extract, Ceritaurea cyanus flower extract, Centaurea cyanus flower distillate, Eucalyptus globulus extract, Saxifrage extract, Lilium (lily) extract, Coix seed extract, oil soluble Coix seed extract, Artemisia princeps pampanini extract, Artemisia princeps pampanini water, Lavandula angustifolia (lavender) extract, Lavandula angustifolia (lavender) water, apple extract, Ganoderma lucidum extract, Lactuca sativa (lettuce) extract, lemon extract, Astragalus sinicus extract, Rosa centifolia (rose) flower water, Rosemarinus officinalis (rosemary) extract, oil soluble Rosemarinus officinalis (rosemary) extract, Anthemis nobilis extract and Sanguisorba officinalis extract;

amino acids and peptides such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, DL-pyrrolidonecarboxylic acid, ε-aminocaproic acid, hydrolyzed elastin, water soluble elastin, hydrolyzed collagen, water soluble collagen, casein, glutathione, wheat peptides and soybean peptide;

vitamins and factors acting like a vitamin such as: vitamin A and analogues thereof such as retinol, retinal, retinoic acid, retinol acetate and retinol palmitate; carotenoids such as α-carotene, β-carotene, γ-carotene, δ-carotene, lycopene, zeaxanthin, cryptoxanthin, echinenon and astaxanthin; vitamin B₁ and analogues thereof such as thiamines; vitamin B₂ and analogues thereof such as riboflavin; vitamin B₆ and analogues thereof such as pyridoxine, pyridoxal and pyridoxamine; vitamin B₁₂ and analogues thereof such as cyanocobalamin; folic acids, nicotinic acid, nicotinamide, pantothenic acids, biotins; vitamin C and analogues thereof such as L-ascorbic acid, sodium L-ascorbate, L-ascorbyl stearate, L-ascorbyl palmitate, L-ascorbyl dipalmitate, L-ascorbyl tetraisopalmitate, disodium L-ascorbate sulfate ester, magnesium L-ascorbyl, sodium L-ascorbyl phosphate, L-ascorbate-2-glucoside and magnesium L-ascorbate-2-phosphate; vitamin D and analogues thereof such as ergocalciferol and cholecalciferol; vitamin E and analogues thereof such as d-α-tocopherol, DL-α-tocopherol, dl-α-tocopherol acetate, dl-α-tocopherol succinate, β-tocopherol, γ-tocopherol and d-δ-tocopherol; ubiquinones, vitamin K and analogues thereof, carnitine, ferulic acid, γ-oryzanol, α-lipoic acid and orotic acid;

antiseptic agents such as benzoic acid, sodium benzoate, undecylenic acid, salicylic acid, sorbic acid, potassium sorbate, dehydroacetic acid, sodium dehydroacetate, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, sodium paraoxybenzoate methyl, phenoxyethanol, light sensitive dye No. 101, light sensitive dye No. 201 and light sensitive dye No. 401;

antioxidizing agents such as butylhydroxyanisole, butylhydroxytoluene, propyl gallate, erythorbic acid, sodium erythorbate, para-hydroxyanisole and octyl gallate;

chelating agents to bind to a metal ion such as trisodium ethylenediamine hydroxyethyl triacetate, edetic acid, disodiumedetate, trisodiumedetate, tetrasodiumedetate, sodiumcitrate, gluconicacid, phytic acid, sodium polyphosphate and sodium metaphosphate;

moisturizing agents such as hyaluronic acid, sodium hyaluronate, sodium chondroitin sulfate, sodium lactate, sodium pyrrolidone carboxylate, betaine, lactic acid bacteria fermented solution, yeast extract and ceramide;

anti-inflammatory agents such as glycyrrhizic acid, trisodium glycyrrhizinate, dipotassium glycyrrhizinate, monoammonium glycyrrhizinate, β-glycyrrhetinic acid, glycerin glycyrrhetinate, stearyl glycyrrhetinate, lysozyme chloride, hydrocortisone and allantoin;

pH adjusting agents such as sodiumhydroxide, potassiumnhydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placenta extract;

essential oils such as Archangelica officinalis (angelica) oil, Canangium odoratum (ylang ylang) oil, Canarium luzonicum (elemi) oil, orange oil, Chamomilla recutita (matricaria) oil, Anthemis nobilis oil, Elettaria cardamom (cardamon) oil, Acorus calamus (calamus) oil, Ferula galbaniflua (galbanum) oil, Cinnamomum camphora (camphor) oil, Daucus carota (carrot) seed oil, Salvia sclarea (clarysage) oil, Citrus paradisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedarwood) oil, Cympogon nardus (citronella) oil, Cinnamomum zeylanicum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium grabeolens (geranium) oil, Thymus vulgaris (thyme) oil, Melaleuca alternifolia (tea tree) oil, Myristica fragrans (nutmeg) oil, Melaleuca qui. viridiflara (niaouli) oil, Citrus aurantium (neroli) oil, pine oil, Ocimum basilicum (basil) oil, Mentha arvensisoil Pogostemon patchouli (patchouli) oil, Cymbopogon martini (palmarosa) oil, Foeniculum vulgare (fennel) oil, Citrus bigaradia (petitgrain) oil, Piper nigrum (black pepper) oil, Boswellia carterii (frankincense) oil, Vetiveria zizanoides (vetivert) oil, Mentha piperita (peppermint) oil, Citrus bergamia (bergamot) oil, benzoin oil, Aniba rosaeodora (bois de rose) oil, Origanum majorana (marjoram) oil, mandarin oil, Conumiphora myrrha (myrrh) oil, Melissa officinalis (balm mint) oil, Eucalyptus globulus oil, Citrus junos oil, Citrus aurantifolia (lime) oil, Ravensare aromaticum (ravensare) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosemarinus officinalis (rosemary) oil and Levisticum officinale (lovage) oil;

terpenes such as limonene, pinene, terpinene, terpinolene, myrcene and longifeelene;

fragrance, water, and the like.

Furthermore, to the external preparation for skin of the invention may also be added any existing raw material of cosmetics at a general concentration. Any of raw materials of cosmetics described in, for example, Keshouhin genryou kizyun (Standards of raw materials of cosmetics), second edition, notes, edited by Society of Japanese Pharmacopoeia, 1984 (YAKUJI NIPPO LIMITED.), Keshouhin genryou kizyun-gai seibun kikaku (Standards of raw materials of cosmetics, nonstandard ingredients), under the editorship of Pharmaceutical Affairs Bureau Evaluation and Registration Division, 1993 (YAKUJI NIPPO LIMITED.) , Keshouhin genryou kizyun-gai seibun kikaku tsuiho (Standards of raw materials of cosmetics, nonstandard ingredient Supplement), under the editorship of Pharmaceutical Affairs Bureau Evaluation and Registration Division, 1993 (YAKUJINIPPOLIMITED.) , Keshouhin syubetsu kyoka kizyun (Standards of cosmetic classification permission), under the editorship of Pharmaceutical Affairs Bureau Evaluation and Registration Division, 1993 (YAKUJINIPPOLIMITED.) , Keshouhin syubetsu haigou seibun kikaku (Standards of cosmetic classification ingredients), under the editorship of Pharmaceutical Affairs Bureau Evaluation and Registration Division, 1997 (YAKUJI NIPPO LIMITED.), Keshouhin genryou jiten (Dictionary of raw materials of cosmetics), 1991 (Nikko Chemicals Co., Ltd.), .), Atarashii Keshouhinkinou Sozai 300 (New 300raw materials having cosmetic functions), 2002 (CMC Publishing Co., Ltd) and the like may be used.

### BEST MODE FOR CARRYING OUT THE INVENTION

Hereunder, the present invention will be specifically explained with reference to the Examples, however, the present invention is not limited to these Examples.

### EXAMPLE 1

Under a nitrogen atmosphere, 1,6-anhydro-β-D-glucopyranose (manufactured by Tokyo Kasei Co., Ltd., 13.0 g), dried propylene carbonate (manufactured by Aldrich Co., 2.5 ml) and 66% by mass of 2-butynyl-tetramethylene sulfonium hexafluoroantimonate (manufactured by Asahi Denka Co., Ltd., 65.8 μl) were put into a schlenk tube which had been thoroughly dried. Then, this schlenk tube was placed in an oil bath, and the oil bath was heated to 100° C., whereby 1,6-anhydro-β-D-glucopyranose was well dissolved. Then, the temperature was raised up to 130° C., whereby polymerization was started while well stirring the mixture. After a 30-minute reaction time, the polymerization solution was poured into methanol to stop the polymerization. After the solvent was distilled off, reprecipitation was repeated with water and methanol. Further, dialysis was performed for purification, and by freeze-drying, a white powdery multi-branched polysaccharide A (5.3 g) was obtained. The yield was 41%. The structure of this multi-branched polysaccharide A was confirmed by ¹H-NHR and ¹³C-NMR. In addition, the weight average molecular weight was 70600 (the light scattering method) and the branching degree was 0.38.

Then, under a nitrogen atmosphere, the multi-branched polysaccharide A (1.0 g) and dried pyridine were put into a flask which had been thoroughly dried. There to was gradually added dropwise leucine ethyl ester isocyanate (2.3 g), and the mixture was reacted at 100° C. for 24 hours. The reaction solution was poured into methanol, the solvent was distilled off, and reprecipitation was repeated with water and methanol. Further, dialysis was performed for purification, and by freeze-drying, a white powder of multi-branched polysaccharide derivative B (1.96 g) was obtained. As a result of an elemental analysis, the substitution degree was 1.4.

Calculation of the substitution degree: Assuming that the substitution degree was 0 when all hydroxyl groups of the multi-branched polysaccharide were free and that the substitution degree was 3 when all hydroxyl groups of the multi-branched polysaccharide were modified, the substitution degree was calculated through values obtained by the elemental analysis on carbon, nitrogen and oxygen, based on the fact that nitrogen was contained only in the leucine ethyl ester isocyanate.

### EXAMPLE 2

The multi-branched polysaccharide derivative B (9.2 mg) obtained in Example 1 was put into a ground-glass Erlenmeyer flask, and dissolved in chloroform (2 ml). Thereto was added rose bengal (21.7 mg), then the flask was sealed hermetically and shaken at 37° C. for 24 hours. After 24 hours, the solution was cooled down to a room temperature, and filtered with a membrane filter with a pore size of 0.45 mm, whereby a solution of a multi-branched polysaccharide/rose bengal complex was obtained. The amount of rose bengal in this multi-branched polysaccharide/rose bengal complex was measured by measuring absorbance of the maximum absorption wavelength, 565 nm, of the diluted solution at a dilution ratio of dimethyl sulfoxide/filtrate=7/1. As a result, in the multi-branched polysaccharide/rose bengal complex, 17 molecules of rose bengal were supplemented in one molecule of the multi-branched polysaccharide. It was demonstrated that rose bengal had been incorporated in the water-soluble portion of the multi-branched polysaccharide.

### EXAMPLE 3

The solvent in the solution of the multi-branched polysaccharide/rose bengal complex obtained in Example 2 was distilled off under reduced pressure, and a particle of the multi-branched polysaccharide/rose bengal complex (the content of rose bengal was 9.7%) was obtained. Ion-exchange water (50 ml) was put into a jar with a lid, and a dialysis membrane (with a cut-off molecular weight of 1000) containing the particle of the multi-branched polysaccharide/rose bengal complex (5.5 mg) was put in the jar and sealed hermetically and the jar was shaken at 37° C. At a regular time interval, 3 ml of the ion-exchange water in the jar was collected, and the eluted amount of rose bengal in water was measured by measuring absorbance of the maximum absorption wavelength, 549 nm, of rose bengal. The eluted amount after 137 hours was 8.8%. It was demonstrated that by exposing the multi-branched polysaccharide/rose bengal complex to water, the rose bengal incorporated in the water-soluble portion of the multi-branched polysaccharide was eluted in water.

Further, sodium hydroxide was added to this water so as to yield a final concentration of 0.1 M. At a regular time interval, 3 ml of the ion-exchange water in the jar was collected, and the eluted amount of rose bengal in water was measured by measuring absorbance of the maximum absorption wavelength, 549 nm, of rose bengal. As a result, the elution rate of rose bengal was increased and after 450 hours, all of the rose bengal included in the multi-branched polysaccharide/rose bengal complex was eluted in the aqueous solution. From this result, it was demonstrated that by changing the environment to an alkali state, the separation rate of rose bengal from the multi-branched polysaccharide/rose bengal complex can be increased.

### EXAMPLE 4

The multi-branched polysaccharide derivative B (9.6 mg) obtained in Example 1 was put into a ground-glass Erlenmeyer flask, and dissolved in chloroform (2 ml). Thereto was added thymol blue (21.3 mg), and then the flask was sealed hermetically and shaken at 37° C. for 24 hours. After 24 hours, the solution was cooled down to a room temperature, and filtered with a membrane filter with a pore size of 0.45 mm, whereby a solution of a multi-branched polysaccharide/thymol blue complex was obtained. The amount of thymol blue in this multi-branched polysaccharide/thymol blue complex was measured by measuring absorbance of the maximum absorption wavelength, 397 nm, of the diluted solution at a dilution ratio of dimethyl sulfoxide/filtrate=7/1. As a result, in the multi-branched polysaccharide/thymol blue complex, 23 molecules of thymol blue were supplemented in one molecule of the multi-branched polysaccharide. It was demonstrated that thymol blue had been incorporated in the water-soluble portion of the multi-branched polysaccharide.

### EXAMPLE 5

The multi-branched polysaccharide derivative B (10.3 mg) obtained in Example 1 was put into a ground-glass Erlenmeyer flask, and dissolved in chloroform (2 ml). Thereto was added methyl orange (20.6 mg), and then the flask was sealed hermetically and shaken at 37° C. for 24 hours. After 24 hours, the solution was cooled down to a room temperature, and filtered with a membrane filter with a pore size of 0.45 mm, whereby a solution of a multi-branched polysaccharide/methyl orange complex was obtained. The amount of methyl orange in this multi-branched polysaccharide/methyl orange complex was measured by measuring absorbance of the maximum absorption wavelength, 423 nm, of the diluted solution at a dilution ratio of dimethyl sulfoxide/filtrate=1/1. As a result, in the multi-branched polysaccharide/methyl orange complex, 3 molecules of methyl orange were incorporated per one molecule of the multi-branched polysaccharide. It was demonstrated that methyl orange had been incorporated in the water-soluble portion of the multi-branched polysaccharide.

### EXAMPLE 6

The multi-branched polysaccharide derivative B (11.8 mg) obtained in Example 1 was put into a ground-glass Erlenmeyer flask, and dissolved in chloroform (2 ml). Thereto was added alizarin yellow (21.3 mg), and then the flask was sealed hermetically and shaken at 37° C. for 24 hours. After 24 hours, the solution was cooled down to a room temperature, and filtered with a membrane filter with a pore size of 0.45 mm, whereby a solution of a multi-branched polysaccharide/alizarin yellow complex was obtained. The amount of alizarin yellow in this multi-branched polysaccharide/alizarin yellow complex was measured by measuring absorbance of the maximum absorption wavelength, 366 nm, of the diluted solution at a dilution ratio of dimethyl sulfoxide/filtrate=1/1. As a result, in the multi-branched polysaccharide/alizarin yellow complex, 8 molecules of alizarin yellow were incorporated per one molecule of the multi-branched polysaccharide. It was demonstrated that alizarin yellow had been incorporated in the water-soluble portion of the multi-branched polysaccharide.

### EXAMPLE 7

The multi-branched polysaccharide A (500 mg) obtained in Example 1, magnesium ascorbic acid 2-phosphate (hereinafter referred to as APM) (500 mg) and water (5 ml) were put into a beaker and dissolved by well stirring. The solution was freeze-dried, where by a white powder of multi-branched polysaccharide-APM complex C (1.0 g) was obtained.

### EXAMPLE 8

Skin lotions shown in Table 1 were prepared based on a conventional method. 10 females of age 20-30 used these lotions, and their feedbacks are shown in Table 2. It was demonstrated that with regard to the sample 1-1 containing the multi-branched polysaccharide-APM complex C where no insoluble components were present, a smooth feeling was maintained. On the other hand, with regard to the sample 1-2, a rough feeling was prominent.

TABLE 1

Sample

| | 1—1 | 1-2 |
|---|---|---|
| Multi-branched polysaccharide- | 20.00% | — |
| APM complex C | | |
| APM | — | 10.00% |
| 1,3-butylene glycol | 5.00% | 5.00% |
| Ethyl alcohol | 5.00% | 5.00% |
| Citric acid | 0.01% | 0.01% |
| Sodium citrate | 1.00% | 1.00% |
| Methyl paraben | 0.20% | 0.20% |
| Purified water | 68.79% | 78.79% |
| | 100.00% | 100.00% |

TABLE 2

Sample

| | 1—1 | 1-2 |
|---|---|---|
| Smooth feeling | 9 | 2 |
| Rough feeling | 1 | 8 |

### EXAMPLE 9

The multi-branched polysaccharide derivative B (500 mg) obtained in Example 1, APM (500 mg), methanol (5.0 ml) and water (25.0 ml) were put into a beaker and dissolved by well stirring. The solution was freeze-dried, whereby a white powder of multi-branched polysaccharide derivative-APM complex D (1.0 g) was obtained.

### EXAMPLE 10

Emulsions shown in Table 3 were prepared based on a conventional methods. 10 females of age 20-30 used these emulsions, and their feedbacks are shown in Table 4. It was demonstrated that with regard to the sample 2-1 containing the multi-branched polysaccharide derivative-APM complex D where no insoluble components were present, a smooth feeling was maintained. On the other hand, with regard to the sample 2-2, a rough feeling was extremely prominent.

TABLE 3

Sample

| | 2-1 | 2—2 |
|---|---|---|
| Multi-branched polysaccharide | 20.0% | — |
| derivative-APM complex D | | |
| APM | — | 10.0% |
| Hydroxyethyl cellulose | 0.5% | 0.5% |
| Xanthan gum | 0.5% | 0.5% |
| Glycerin | 8.0% | 8.0% |
| 1,3-butylene glycol | 2.0% | 2.0% |
| Sodium citrate | 1.0% | 1.0% |
| Polyethylene glycol-50 | 0.5% | 0.5% |
| Methylparaben | 0.2% | 0.2% |
| Purified water | 67.3% | 77.3% |
| | 100.0% | 100.0% |

TABLE 4

Sample

| | 2-1 | 2—2 |
|---|---|---|
| Smooth feeling | 8 | 0 |
| Rough feeling | 2 | 10 |

### INDUSTRIAL APPLICABILITY

The present invention provides a complex of a multi-branched polysaccharide derivative and a functional compound, which can control dissolution and elution of the functional compound.

According to the present invention, the functional compound can be efficiently incorporated into polysaccharide derivative, and further by exposing the polysaccharide complex to an environment such as a solution, sustained release of the functional compound can be controlled. By utilizing this property, the functional compound can be efficiently used.

For example, by conjugating a pigment compound, the complex can be used as a make-up cosmetic product, colored gel, color filter, polarizing filter, water-soluble capsule, coloring material in paint, ink-jet ink, fiber dyeing, prolonged radical trapping agent, prolonged radical generating agent or the like. In addition, the complex can be used, for example, for stabilizing and improving solubility of a natural extract in a cosmetic product by combining the complex, for stabilizing vitamins, improving the solubility of vitamins or prolonging a sustained release effect of a quasi-drug product or a drug product by combining the complex, for maintaining sustained release of drug in a cataplasm, for imparting sustained release of drug in an adhesive bandage, or the like. Further, with regard to a general physiologically active substance, by conjugation, the complex can be used for a sustained release fertilizer, a sustained release agricultural chemical or the like.

1. A polysaccharide/functional compound complex comprising a functional compound and a multi-branched polysaccharide derivative, wherein the multi-branched polysaccharide derivative has a skeleton of a multi-branched polysaccharide composed of a saccharide as a structural unit, and at least one of the hydroxyl groups (OH) of the multi-branched polysaccharide is substituted by OR, wherein R represents a hydrogen atom, a hydrocarbon having 1 to 30 carbon atoms, or a hydrocarbon having 1 to 30 carbon atoms and hetero atom.

2. The polysaccharide/functional compound complex according to claim 1, wherein the multi-branched polysaccharide constituting the skeleton is a polymer composed of, as a monomer, an anhydrosaccharide. and/or an anhydrosaccharide derivative in which at least one of the hydroxyl groups (OH) of the anhydrosaccharide has been substituted by OR, wherein R represents a hydrogen atom, a hydrocarbon having 1 to 30 carbon atoms, or a hydrocarbon having 1 to 30 carbon atoms and hetero atom.

3. The polysaccharide/functional compound complex according to claim 2, wherein the anhydrosaccharide or the anhydrosaccharide portion of the anhydrosaccharide derivative is at least one kind selected from the group consisting of 1,6-anhydrosaccharide, 1,4-anhydrosaccharide, 1,3-anhydrosaccharide, 1,2-anhydrosaccharide, and 5,6-anhydrosaccharide.

4. The polysaccharide/functional compound complex according to claim 1, wherein the branching degree of the multi-branched polysaccharide is 0.05 to 1.00.

5. The polysaccharide/functional compound complex according to claim 1, wherein the functional compound is a pigment compound.

6. The polysaccharide/functional compound complex according to any claim 1, wherein the functional compound is a compound having a physiological activity.

7. The polysaccharide/functional compound complex according to claim 6, wherein the compound having a physiological activity is at least one selected from the group consisting of raw material compounds for cosmetic products, raw material compounds for quasi-drug products and compounds for drug products.

8. The polysaccharide/functional compound complex according to claim 7, wherein the raw material compound for a cosmetic product is ascorbic acid, an ascorbate or an ascorbic acid derivative.

9. The polysaccharide/functional compound complex according to claim 2, wherein the branching degree of the multi-branched polysaccharide is 0.05 to 1.00.

10. The polysaccharide/functional compound complex according to claim 2, wherein the functional compound is a pigment compound.

11. The polysaccharide/functional compound complex according to claim 2, wherein the functional compound is a compound having a physiological activity.

12. The polysaccharide/functional compound complex according to claim 11, wherein the compound having a physiological activity is at least one selected from the group consisting of raw material compounds for cosmetic products, raw material compounds for quasi-drug products and compounds for drug products.

13. The polysaccharide/functional compound complex according to claim 12, wherein the raw material compound for a cosmetic product is ascorbic acid, an ascorbate or an ascorbic acid derivative.

14. The polysaccharide/functional compound complex according to claim 4, wherein the functional compound is a pigment compound.

15. The polysaccharide/functional compound complex according to claim 14, wherein the functional compound is a compound having a physiological activity.

16. The polysaccharide/functional compound complex according to claim 15, wherein the compound having a physiological activity is at least one selected from the group consisting of raw material compounds for cosmetic products, raw material compounds for quasi-drug products and compounds for drug products.

17. The polysaccharide/functional compound complex according to claim 16, wherein the raw material compound for a cosmetic product is ascorbic acid, an ascorbate or an ascorbic acid derivative.

18. An external preparation for skin including 0.01 to 100% by mass of the polysaccharide/functional compound complex according to claim 1.

19. A cosmetic product including 0.01 to 100% by mass of the polysaccharide/functional compound complex according to claim 1.
